(19) United States
(12) Patent Application Publication
(10) Pub. No.: US 2021/0025875 A1
(43) Pub. Date: Jan. 28, 2021

(54) USE OF INSULIN-LIKE GROWTH FACTOR-BINDING PROTEIN 7 AND TISSUE INHIBITOR OF METALLOPROTEINASE 2 IN THE MANAGEMENT OF RENAL REPLACEMENT THERAPY

(71) Applicant: ASTUTE MEDICAL, INC., SAN DIEGO, CA (US)

(72) Inventors: PAUL MCPHERSON, ENCINITAS, CA (US); JAMES PATRICK KAMPF, SAN DIEGO, CA (US)

(73) Assignee: ASTUTE MEDICAL, INC., SAN DIEGO, CA (US)

(21) Appl. No.: 16/611,456

(22) PCT Filed: May 7, 2018
(86) PCT No.: PCT/US2018/031425

§ 371 Date: Nov. 6, 2019

Related U.S. Application Data
(60) Provisional application No. 62/502,728, filed on May 7, 2017.

(51) Int. Cl. G01N 33/543

(52) U.S. Cl. CPC G01N 33/543; G01N 2333/8146; A61K 45/06; G01N 2800/52; G01N 2333/4745; G01N 2800/347

(57) ABSTRACT

The present invention provides methods and compositions for managing renal replacement therapy. A risk score, which is determined from a urinary concentration of IGFBP7 (insulin-like growth factor-binding protein 7) and/or a urinary concentration of TIMP-2 (tissue inhibitor of metalloproteinase 2), is determined obtained from the patient, and is used to manage patient treatment.

### CROSS-REFERENCE TO RELATED APPLICATIONS

The present application claims the benefit of U.S. Provisional Application No. 62/502,728, filed May 7, 2017, which is hereby incorporated by reference in its entirety including all tables, figures and claims.

### BACKGROUND OF THE INVENTION

The following discussion of the background of the invention is merely provided to aid the reader in understanding the invention and is not admitted to describe or constitute prior art to the present invention.C

The kidney is responsible for water and solute excretion from the body. Its functions include maintenance of acid-base balance, regulation of electrolyte concentrations, control of blood volume, and regulation of blood pressure. As such, loss of kidney function through injury and/or disease results in substantial morbidity and mortality. A detailed discussion of renal injuries is provided in Harrison's Principles of Internal Medicine, 17^{th} Ed., McGraw Hill, New York, pages 1741-1830, which are hereby incorporated by reference in their entirety. Renal disease and/or injury may be acute or chronic. Acute and chronic kidney disease are described as follows (from Current Medical Diagnosis & Treatment 2008, 47^{th} Ed, McGraw Hill, New York, pages 785-815, which are hereby incorporated by reference in their entirety): “Acute renal failure is worsening of renal function over hours to days, resulting in the retention of nitrogenous wastes (such as urea nitrogen) and creatinine in the blood. Retention of these substances is called azotemia. Chronic renal failure (chronic kidney disease) results from an abnormal loss of renal function over months to years”.

Acute renal failure (ARF, also known as acute kidney injury, or AKI) is an abrupt (typically detected within about 48 hours to 1 week) reduction in glomerular filtration. This loss of filtration capacity results in retention of nitrogenous (urea and creatinine) and non-nitrogenous waste products that are normally excreted by the kidney, a reduction in urine output, or both. It is reported that ARF complicates about 5% of hospital admissions, 4-15% of cardiopulmonary bypass surgeries, and up to 30% of intensive care admissions. ARF may be categorized as prerenal, intrinsic renal, or postrenal in causation. Intrinsic renal disease can be further divided into glomerular, tubular, interstitial, and vascular abnormalities. Major causes of ARF are described in the following table, which is adapted from the Merck Manual, 17^{th} ed., Chapter 222, and which is hereby incorporated by reference in their entirety:

| Type | Risk Factors |
|---|---|
| Prerenal | |
| ECF volume depletion | Excessive diuresis, hemorrhage, GI losses, loss of |
| | intravascular fluid into the extravascular space (due to |
| | ascites, peritonitis, pancreatitis, or burns), loss of skin |
| | and mucus membranes, renal salt- and water-wasting |
| | states |
| Low cardiac output | Cardiomyopathy, MI, cardiac tamponade, pulmonary |
| | embolism, pulmonary hypertension, positive-pressure |
| | mechanical ventilation |
| Low systemic vascular resistance | Septic shock, liver failure, antihypertensive drugs |
| Increased renal vascular resistance | NSAIDs, cyclosporines, tacrolimus, hypercalcemia, |
| | anaphylaxis, anesthetics, renal artery obstruction, renal |
| | vein thrombosis, sepsis, hepatorenal syndrome |
| Decreased efferent arteriolar tone | ACE inhibitors or angiotensin II receptor blockers |
| (leading to decreased GFR from | |
| reduced glomerular transcapillary | |
| pressure, especially in patients | |
| with bilateral renal artery stenosis) | |
| Intrinsic Renal | |
| Acute tubular injury | Ischemia (prolonged or severe prerenal state): surgery, |
| | hemorrhage, arterial or venous obstruction; Toxins: |
| | NSAIDs, cyclosporines, tacrolimus, aminoglycosides, |
| | foscarnet, ethylene glycol, hemoglobin, myoglobin, |
| | ifosfamide, heavy metals, methotrexate, radiopaque |
| | contrast agents, streptozotocin |
| Acute glomerulonephritis | ANCA-associated: Crescentic glomerulonephritis, |
| | polyarteritis nodosa, Wegener's granulomatosis; Anti- |
| | GBM glomerulonephritis: Goodpasture's syndrome; |
| | Immune-complex: Lupus glomerulonephritis, |
| | postinfectious glomerulonephritis, cryoglobulinemic |
| | glomerulonephritis |
| Acute tubulointerstitial nephritis | Drug reaction (eg, β-lactams, NSAIDs, sulfonamides, |
| | ciprofloxacin, thiazide diuretics, furosemide, phenytoin, |
| | allopurinol, pyelonephritis, papillary necrosis |
| Acute vascular nephropathy | Vasculitis, malignant hypertension, thrombotic |
| | microangiopathies, scleroderma, atheroembolism |
| Infiltrative diseases | Lymphoma, sarcoidosis, leukemia |
| Postrenal | |
| Tubular precipitation | Uric acid (tumor lysis), sulfonamides, triamterene, |
| | acyclovir, indinavir, methotrexate, ethylene glycol |
| | ingestion, myeloma protein, myoglobin |
| Ureteral obstruction | Intrinsic: Calculi, clots, sloughed renal tissue, fungus |
| | ball, edema, malignancy, congenital defects; Extrinsic: |
| | Malignancy, retroperitoneal fibrosis, ureteral trauma |
| | during surgery or high impact injury |
| Bladder obstruction | Mechanical: Benign prostatic hyperplasia, prostate |
| | cancer, bladder cancer, urethral strictures, phimosis, |
| | paraphimosis, urethral valves, obstructed indwelling |
| | urinary catheter; Neurogenic: Anticholinergic drugs, |
| | upper or lower motor neuron lesion |

A commonly reported criteria for defining and detecting AKI is an abrupt (typically within about 2-7 days or within a period of hospitalization) elevation of serum creatinine. Although the use of serum creatinine elevation to define and detect AKI is well established, the magnitude of the serum creatinine elevation and the time over which it is measured to define AKI varies considerably among publications. Traditionally, relatively large increases in serum creatinine such as 100%, 200%, an increase of at least 100% to a value over 2 mg/dL and other definitions were used to define AKI. However, the recent trend has been towards using smaller serum creatinine rises to define AKI. The relationship between serum creatinine rise, AKI and the associated health risks are reviewed in Praught and Shlipak, Curr Opin Nephrol Hypertens 14:265-270, 2005 and Chertow et al, J Am Soc Nephrol 16: 3365-3370, 2005, which, with the references listed therein, are hereby incorporated by reference in their entirety. As described in these publications, acute worsening renal function (AKI) and increased risk of death and other detrimental outcomes are now known to be associated with very small increases in serum creatinine. These increases may be determined as a relative (percent) value or a nominal value. Relative increases in serum creatinine as small as 20% from the pre-injury value have been reported to indicate acutely worsening renal function (AKI) and increased health risk, but the more commonly reported value to define AKI and increased health risk is a relative increase of at least 25%. Nominal increases as small as 0.3 mg/dL, 0.2 mg/dL or even 0.1 mg/dL have been reported to indicate worsening renal function and increased risk of death. Various time periods for the serum creatinine to rise to these threshold values have been used to define AKI, for example, ranging from 2 days, 3 days, 7 days, or a variable period defined as the time the patient is in the hospital or intensive care unit. These studies indicate there is not a particular threshold serum creatinine rise (or time period for the rise) for worsening renal function or AKI, but rather a continuous increase in risk with increasing magnitude of serum

In an effort to reach consensus on a unified classification system for using serum creatinine to define AKI in clinical trials and in clinical practice, Bellomo et al., Crit Care. 8(4):R204-12, 2004, which is hereby incorporated by reference in its entirety, proposes the following classifications for stratifying AKI patients:

“Risk”: serum creatinine increased 1.5 fold from baseline OR urine production of <0.5 ml/kg body weight/hr for 6 hours;

“Injury”: serum creatinine increased 2.0 fold from baseline OR urine production <0.5 ml/kg/hr for 12 h;

“Failure”: serum creatinine increased 3.0 fold from baseline OR creatinine >355 μmol/l (with a rise of >44) or urine output below 0.3 ml/kg/hr for 24 h or anuria for at least 12 hours;

And included two clinical outcomes:

“Loss”: persistent need for renal replacement therapy for more than four weeks.

“ESRD”: end stage renal disease—the need for dialysis for more than 3 months.

These criteria are called the RIFLE criteria, which provide a useful clinical tool to classify renal status. As discussed in Kellum, Crit. Care Med. 36: S141-45, 2008 and Ricci et al., Kidney Int. 73, 538-546, 2008, each hereby incorporated by reference in its entirety, the RIFLE criteria provide a uniform definition of AKI which has been validated in numerous studies.

More recently, Mehta et al., Crit. Care 11:R31 (doi:10.1186.cc5713), 2007, hereby incorporated by reference in its entirety, proposes the following similar classifications for stratifying AKI patients, which have been modified from RIFLE:

“Stage I”: increase in serum creatinine of more than or equal to 0.3 mg/dL (≥26.4 μmol/L) or increase to more than or equal to 150% (1.5-fold) from baseline OR urine output less than 0.5 mL/kg per hour for more than 6 hours;

“Stage II”: increase in serum creatinine to more than 200% (>2-fold) from baseline OR urine output less than 0.5 mL/kg per hour for more than 12 hours;

“Stage III”: increase in serum creatinine to more than 300% (>3-fold) from baseline OR serum creatinine ≥354 μmol/L accompanied by an acute increase of at least 44 μmol/L OR urine output less than 0.3 mL/kg per hour for 24 hours or anuria for 12 hours.

Likewise, Kidney Disease: Improving Global Outcomes (KDIGO) Acute Kidney Injury Work Group. KDIGO Clinical Practice Guideline for Acute Kidney Injury, Kidney inter., Suppl. 2012; 2: 1-138, refers to both RIFLE and AKIN, and offers the following AKI staging guidelines:

| Stage | Serum creatinine or | Urine output |
|---|---|---|
| 1 | 1.5-1.9 times baseline | <0.5 ml/kg/h for 6-12 hours |
| | or ≥0.3 mg/dl (≥26.5 mmol/l) | |
| | increase | |
| 2 | 2.0-2.9 times baseline | <0.5 ml/kg/h for ≥12 hours |
| 3 | 3.0 times baseline | <0.3 ml/kg/h for ≥24 hours |
| | Or | or |
| | Increase in serum creatinine | Anuria for >12 hours |
| | to ≥4.0 mg/dl (X353.6 mmol/l) | |
| | or | |
| | Initiation of renal replacement | |
| | therapy or | |
| | In patients <18 years, decrease in | |
| | eGFR to <35 ml/min per 1.73 m² | |

The CIN Consensus Working Panel (McCollough et al, Rev Cardiovasc Med. 2006; 7(4):177-197, hereby incorporated by reference in its entirety) uses a serum creatinine rise of 25% to define Contrast induced nephropathy (which is a type of AKI). Although various groups propose slightly different criteria for using serum creatinine to detect AKI, the consensus is that small changes in serum creatinine, such as 0.3 mg/dL or 25%, are sufficient to detect AKI (worsening renal function) and that the magnitude of the serum creatinine change is an indicator of the severity of the AKI and mortality risk.

In contrast, chronic kidney disease (CKD) is a different clinical entity characterized by irreversible nephron loss. A progressive decline in renal function is observed over a period of months or years with few, if any, symptoms until the chronic injury is far advanced. CKD is characterized histologically by the concurrent development of glomerulosclerosis and tubulointerstitial fibrosis. Podocyte damage and loss has been identified as a key mechanism, at which a number of glomerular pathomechanisms converge to result in glomerulosclerosis. The mesangial cell is the major matrix forming cell in the glomerulus and is also pivotal to the glomerulosclerotic process, while the activated (alpha-smooth muscle actin-positive) interstitial fibroblast or myofibroblast is central to the development of tubulointerstitial fibrosis. In chronic renal failure, the tubules become scarred causing water loss. In contrast to the oliguia seen in AKI, CKD typically results in polyruria (increased urine volume).

The Merck Manual discusses the need to distinguish between acute renal failure and chronic renal disease, as these are different conditions with different therapies (see, inter alia, page 1846, right hand column, section “Diagnosis”, first sentence “the first step is to determine whether the renal failure is acute, chronic or super-imposed on chronic, and Table 222-4 on page 1847 “Classification of Acute Versus Chronic Renal Failure). Recently, a prospective, multicenter investigation in which two novel biomarkers for AKI were identified in a discovery cohort of critically ill adult patients and subsequently validated using a clinical assay and compared to existing markers of AKI in an independent validation cohort of heterogeneous critically ill patients. Urinary insulin-like growth factor binding protein 7 (IGFBP7) and tissue inhibitor of metalloproteinase 2 (TIMP-2) robust markers that have improved performance characteristics when directly compared with existing methods for detecting risk for AKI, but also provide significant additional information over clinical data. It is notable that IGFBP7 and TIMP-2 are each involved with the phenomenon of G₁ cell cycle arrest during the very early phases of cell injury, it has been shown that renal tubular cells enter a short period of G1 cell-cycle arrest following injury from experimental sepsis or ischemia. See, e.g., Yang et al., J. Infect. 58:459-464, 2009; Witzgall et al., J. Clin. Invest. 93:2175-2188, 1994.

### BRIEF SUMMARY OF THE INVENTION

It is an object of the present invention to provide methods and compositions for guiding the use of renal replacement therapy in patients.

In a first aspect, the present invention relates to methods for managing a patient in need of renal replacement therapy, comprising:

calculating a risk score which is (i) a urinary concentration of IGFBP7 (insulin-like growth factor-binding protein 7), (ii) a urinary concentration of TIMP-2 (tissue inhibitor of metalloproteinase 2), or (iii) a composite of a urinary concentration of IGFBP7 and a urinary concentration of TIMP-2, by measuring an IGFBP7 concentration and/or a TIMP-2 concentration in a urine sample obtained from the subject to provide the risk score;

comparing the risk score to a risk score threshold value, wherein when the risk score is above the risk score threshold value the subject is determined to be in renal stress; and

if the comparing step indicates that the subject is in renal stress, treating the subject with a method of renal replacement therapy that produces less renal stress relative to treatment with intermittent hemodialysis.

In certain embodiments, the method of renal replacement therapy that produces less renal stress relative to treatment with intermittent hemodialysis is continuous renal replacement therapy or prolonged intermittent renal replacement therapy (PIRRT). PIRRT as used herein includes sustained low efficiency (daily) dialysis (SLEDD), sustained low efficiency (daily) diafiltration (SLEDD-f), extended daily dialysis (EDD), slow continuous dialysis (SCD), go slow dialysis, and accelerated venovenous hemofiltration (AVVH).

In certain embodiments, the risk score is calculated by the use of a mathematical function that includes each of an IGFBP7 and TIMP-2 concentration in the calculation of the function. By way of example, the risk score may be calculated by multiplication of the concentrations of IGFBP7 and TIMP-2. In preferred embodiments, the risk score is ([TIMP-2]×[IGFBP7])/1000, where the concentrations of IGFBP7 and TIMP-2 are each measured in ng/mL.

In certain exemplary embodiments, the risk score is ([TIMP-2]×[IGFBP7])/1000, where the concentrations of IGFBP7 and TIMP-2 are each measured in ng/mL, and the threshold value is about 2.0. In other exemplary embodiments, the risk score is [TIMP-2] measured in ng/mL and the threshold is about 12.0. In still other exemplary embodiments, the risk score is [IGFBP7] measured in ng/mL and the threshold is about 150.0.

The ability of a particular test to distinguish two populations can be established using ROC analysis. For example, ROC curves established from a “first” subpopulation which is predisposed to one or more future changes in renal status, and a “second” subpopulation which is not so predisposed can be used to calculate a ROC curve, and the area under the curve provides a measure of the quality of the test. Preferably, the tests described herein provide a ROC curve area greater than 0.5, preferably at least 0.6, more preferably 0.7, still more preferably at least 0.8, even more preferably at least 0.9, and most preferably at least 0.95.

In certain aspects, the measured IGFBP7 and/or TIMP-2 concentrations may be treated as continuous variables. For example, any particular concentration can be converted into a corresponding probability of a future reduction in renal function for the subject, the occurrence of an injury, a classification, etc. In yet another alternative, a threshold that can provide an acceptable level of specificity and sensitivity in separating a population of subjects into “bins” such as a “first” subpopulation (e.g., which is predisposed to one or more future changes in renal status, the occurrence of an injury, a classification, etc.) and a “second” subpopulation which is not so predisposed. A threshold value is selected to separate this first and second population by one or more of the following measures of test accuracy:

an odds ratio greater than 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less;

a specificity of greater than 0.5, preferably at least about 0.6, more preferably at least about 0.7, still more preferably at least about 0.8, even more preferably at least about 0.9 and most preferably at least about 0.95, with a corresponding sensitivity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

a sensitivity of greater than 0.5, preferably at least about 0.6, more preferably at least about 0.7, still more preferably at least about 0.8, even more preferably at least about 0.9 and most preferably at least about 0.95, with a corresponding specificity greater than 0.2, preferably greater than about 0.3, more preferably greater than about 0.4, still more preferably at least about 0.5, even more preferably about 0.6, yet more preferably greater than about 0.7, still more preferably greater than about 0.8, more preferably greater than about 0.9, and most preferably greater than about 0.95;

at least about 75% sensitivity, combined with at least about 75% specificity;

a positive likelihood ratio (calculated as sensitivity/(1-specificity)) of greater than 1, at least about 2, more preferably at least about 3, still more preferably at least about 5, and most preferably at least about 10; or

a negative likelihood ratio (calculated as (1-sensitivity)/specificity) of less than 1, less than or equal to about 0.5, more preferably less than or equal to about 0.3, and most preferably less than or equal to about 0.1.

The term “about” in the context of any of the above measurements refers to +/−5% of a given measurement.

Multiple thresholds may also be used to assess renal status in a subject. For example, a “first” subpopulation which is predisposed to one or more future changes in renal status, the occurrence of an injury, a classification, etc., and a “second” subpopulation which is not so predisposed can be combined into a single group. This group is then subdivided into three or more equal parts (known as tertiles, quartiles, quintiles, etc., depending on the number of subdivisions). An odds ratio is assigned to subjects based on which subdivision they fall into. If one considers a tertile, the lowest or highest tertile can be used as a reference for comparison of the other subdivisions. This reference subdivision is assigned an odds ratio of 1. The second tertile is assigned an odds ratio that is relative to that first tertile. That is, someone in the second tertile might be 3 times more likely to suffer one or more future changes in renal status in comparison to someone in the first tertile. The third tertile is also assigned an odds ratio that is relative to that first tertile.

In certain embodiments, the urinary concentration of IGFBP7 and/or the urinary concentration of TIMP-2 are measured by introducing the urine sample obtained from the subject into an immunoassay instrument; wherein the immunoassay instrument comprises a solid phase, and one or both of an IGFBP7 antibody immobilized at a first location on the solid phase and a TIMP-2 antibody immobilized at a second location on the solid phase; wherein the instrument causes the urine sample to contact one or both of the first location and the second location. The instrument measures the amount of IGFBP7 which binds to the IGFBP7 antibody immobilized at the first location and determines therefrom the concentration of IGFBP7 in the urine sample; and/or the instrument measures the amount of TIMP-2 which binds to the TIMP-2 antibody immobilized at the second location and determines therefrom the concentration of TIMP-2 in the urine sample.

In certain embodiments, the instrument optionally mathematically combines the concentration of IGFBP7 and the concentration of TIMP-2 in the urine sample into the risk score; and optionally the instrument reports the risk score in a human readable form.

Preferred are sandwich immunoassays. In these embodiments, the urine sample obtained from the patient may be further contacted with a second IGFBP7 antibody conjugated to detectable label and a second TIMP-2 antibody conjugated to detectable label; wherein first sandwich complexes are formed between the IGFBP7 antibody, IGFBP7 present in the urine sample, and the second IGFBP7 antibody; wherein second sandwich complexes are formed between the TIMP-2 antibody, TIMP-2 present in the urine sample, and the second TIMP-2 antibody; wherein the amount of IGFBP7 which binds to the IGFBP7 antibody is determined by the instrument detecting the detectable label bound at the first location; and wherein the amount of TIMP-2 which binds to the TIMP-2 antibody is determined by the instrument detecting the detectable label bound at the second location.

The term “about” as used throughout this document refers to +/−10% of a given value.

Managing the patient based on the calculated risk score comprises treating the subject with a method of renal replacement therapy that reduces renal stress relative to treatment with intermittent hemodialysis. In various embodiments, the patient is an intensive care unit patient; the patient is in acute renal failure; the patient has sepsis; and/or the patient is recovering from surgery.

The use of renal replacement therapy is understood in the art, and may be performed as described in one or more of the following publications, which are hereby incorporated by reference:

Tolwani A J, Wheeler T S, Wille K M. Sustained low-efficiency dialysis. Contrib Nephrol 2007; 156:320.

Naka T, Baldwin I, Bellomo R, et al. Prolonged daily intermittent renal replacement therapy in ICU patients by ICU nurses and ICU physicians. Int J Artif Organs 2004; 27:380.

Bellomo R, Baldwin I, Fealy N. Prolonged intermittent renal replacement therapy in the intensive care unit. Crit Care Resusc 2002; 4:281.

Marshall M R, Golper T, Shaver MJ, Chatoth DK. Hybrid renal replacement modalities for the critically ill. Contrib Nephrol 2001; :252.

Marshall M R, Golper T A. Low-efficiency acute renal replacement therapy: role in acute kidney injury. Semin Dial 2011; 24:142.

Overberger P, Pesacreta M, Palevsky P M, VA/NIH Acute Renal Failure Trial Network. Management of renal replacement therapy in acute kidney injury: a survey of practitioner prescribing practices. Clin J Am Soc Nephrol 2007; 2:623.

Ricci Z, Ronco C, D'Amico G, et al. Practice patterns in the management of acute renal failure in the critically ill patient: an international survey. Nephrol Dial Transplant 2006; 21:690.

Basso F, Ricci Z, Cruz D, Ronco C. International survey on the management of acute kidney injury in critically ill patients: year 2007. Blood Purif 2010; 30:214.

Sigler M H, Teehan B P. Solute transport in continuous hemodialysis: a new treatment for acute renal failure. Kidney Int 1987; 32:562.

Bellomo R. Choosing a therapeutic modality: Hemodialysis vs hemodiafiltration. Semin Dial 1996; 9:88.

Macias W L, Mueller B A, Scarim S K, et al. Continuous venovenous hemofiltration: an alternative to continuous arteriovenous hemofiltration and hemodiafiltration in acute renal failure. Am J Kidney Dis 1991; 18:451.

Kihara M, Ikeda Y, Shibata K, et al. Slow hemodialysis performed during the day in managing renal failure in critically ill patients. Nephron 1994; 67:36.

Hombrouckx R, Bogaert A M, Leroy F, et al. Go-slow dialysis instead of continuous arteriovenous hemofiltration. Contrib Nephrol 1991; 93:149.

Kudoh Y, Iimura O. Slow continuous hemodialysis--new therapy for acute renal failure in critically ill patients--Part 1. Theoretical consideration and new technique. Jpn Circ J 1988; 52:1171.

Kudoh Y, Shiiki M, Sasa Y, et al. Slow continuous hemodialysis--new therapy for acute renal failure in critically ill patients--Part 2. Animal experiments and clinical implication. Jpn Circ J 1988; 52:1183.

Mehta R L, Martin R K. Initiating and implementing a continuous renal replacement therapy program. Semin Dial 1996; 9:80.

Bagshaw et al., Precision Continuous Renal Replacement Therapy and Solute Control. Blood Purif. 2016; 42:238-247.

Murugan et al., Precision Fluid Management in Continuous Renal Replacement Therapy. Blood Purif 2016; 42:266-278.

Ostermann et al., Patient Selection and Timing of Continuous Renal Replacement Therapy. Blood Purif 2016; 42:224-237.

Cerda et al., Role of Technology for the Management of AKI in Critically Ill Patients: From Adoptive Technology to Precision Continuous Renal Replacement Therapy. Blood Purif 2016; 42:248-265.

Kellum and Ronco, The 17th Acute Disease Quality Initiative International Consensus Conference: Introducing Precision Renal Replacement Therapy. Blood Purif 2016; 42:221-223.

Additional clinical indicia of health status, and particularly of renal sufficiency, may be combined with the IGFBP7 and/or TIMP-2 measurements in the methods described herein. Such clinical indicia may include one or more of: a baseline urine output value for the patient, a baseline change in serum creatinine for the patient, demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), other clinical variables (e.g., blood pressure, temperature, respiration rate), risk scores (APACHE score, PREDICT score, TIMI Risk Score for UA/NSTEMI, Framingham Risk Score, risk scores of Thakar et al. (J. Am. Soc. Nephrol. 16: 162-68, 2005), Mehran et al. (J. Am. Coll. Cardiol. 44: 1393-99, 2004), Wijeysundera et al. (JAMA 297: 1801-9, 2007), Goldstein and Chawla (Clin. J. Am. Soc. Nephrol. 5: 943-49, 2010), or Chawla et al. (Kidney Intl. 68: 2274-80, 2005)), a glomerular filtration rate, an estimated glomerular filtration rate, a urine production rate, a serum or plasma creatinine concentration, a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatnine ratio, a renal failure index calculated as urine sodium/(urine creatinine/plasma creatinine), a serum or plasma neutrophil gelatinase (NGAL) concentration, a urine NGAL concentration, a serum or plasma cystatin C concentration, a serum or plasma cardiac troponin concentration, a serum or plasma BNP concentration, a serum or plasma NTproBNP concentration, and a serum or plasma proBNP concentration. Other measures of renal function which may be combined with IGFBP7 and/or TIMP-2 assay result(s) are described hereinafter and in Harrison's Principles of Internal Medicine, 17 th Ed., McGraw Hill, New York, pages 1741-1830, and Current Medical Diagnosis & Treatment 2008, 47 th Ed, McGraw Hill, New York, pages 785-815, each of which are hereby incorporated by reference in their entirety. p The methods described herein can be used at the initiation of renal replacement therapy for the patient, and/or can be used as a monitoring tool for an ongoing renal replacement protocol. Thus, in certain aspects, the patient is undergoing renal replacement therapy at the time the urine sample is obtained from the subject to provide the risk score.

In certain aspects in which the risk score is used to monitor ongoing renal replacement therapy, the risk score may be compared to the threshold, and if the risk score is above the threshold, the rate or amount of fluid volume being removed from the subject by the ongoing renal replacement therapy may be reduced, and/or the clearance rate of solutes by the ongoing renal replacement therapy may be reduced. This clearance rate is often described in terms of “dose,” which identifies the volume of blood cleared of waste products and toxins by the extracorporeal circuit per unit of time. In practice, it is measured as the rate of removal of a representative solute. Urea is the solute most commonly used to quantify dose. Neri et al., Nomenclature for renal replacement therapy in acute kidney injury: basic principles. Critical Care 2016, 20: 318, which is hereby incorporated by reference.

By way of example, monitoring may involve a switch from intermittent hemodialysis to continuous renal replacement therapy or prolonged intermittent renal replacement therapy. Alternatively, it may involve altering the parameters of the renal replacement protocol to reduce hypotensive effects associated with the ongoing renal replacement therapy or reducing dose, e.g., by using variable dialysate sodium profiles (160-140 meq/L), variable ultrafiltration rates, setting dialysate temperature to below 37° C. combined with prolonged treatment time or altered frequency and enable safer treatment.

### DETAILED DESCRIPTION OF THE INVENTION

For purposes of this document, the following definitions apply:

As used herein, an “injury to renal function” is an abrupt (within 14 days, preferably within 7 days, more preferably within 72 hours, and still more preferably within 48 hours) measurable reduction in a measure of renal function. Such an injury may be identified, for example, by a decrease in glomerular filtration rate or estimated GFR, a reduction in urine output, an increase in serum creatinine, an increase in serum cystatin C, a requirement for renal replacement therapy, etc. “Improvement in Renal Function” is an abrupt (within 14 days, preferably within 7 days, more preferably within 72 hours, and still more preferably within 48 hours) measurable increase in a measure of renal function. Preferred methods for measuring and/or estimating GFR are described hereinafter.

As used herein, “reduced renal function” is an abrupt (within 14 days, preferably within 7 days, more preferably within 72 hours, and still more preferably within 48 hours) reduction in kidney function identified by an absolute increase in serum creatinine of greater than or equal to 0.1 mg/dL (≥8.8 _{I}lmol/L), a percentage increase in serum creatinine of greater than or equal to 20% (1.2-fold from baseline), or a reduction in urine output (documented oliguria of less than 0. 5 ml/kg per hour).

As used herein, “acute renal failure” or “ARF” is an abrupt (within 14 days, preferably within 7 days, more preferably within 72 hours, and still more preferably within 48 hours) reduction in kidney function identified by an absolute increase in serum creatinine of greater than or equal to 0.3 mg/dl (≥26.4 μmol/l), a percentage increase in serum creatinine of greater than or equal to 50% (1. 5-fold from baseline), or a reduction in urine output (documented oliguria of less than 0.5 ml/kg per hour for at least 6 hours). This term is synonymous with “acute kidney injury” or “AKI.”

As used herein, chronic kidney disease or “CKD” is CKD is defined as abnormalities of kidney structure or function, present for >3 months, with implications for health. Approximately 11% of U.S. adults reportedly have CKD, many of whom are elderly. The condition is usually asymptomatic until its advanced stages.

The term “subject” as used herein refers to a human or non-human organism. Thus, the methods and compositions described herein are applicable to both human and veterinary disease. Further, while a subject is preferably a living organism, the invention described herein may be used in post-mortem analysis as well. Preferred subjects are humans, and most preferably “patients,” which as used herein refers to living humans that are receiving medical care for a disease or condition. This includes persons with no defined illness who are being investigated for signs of pathology.

Preferably, an analyte is measured in a sample. Such a sample may be obtained from a subject, or may be obtained from biological materials intended to be provided to the subject. For example, a sample may be obtained from a kidney being evaluated for possible transplantation into a subject, and an analyte measurement used to evaluate the kidney for preexisting damage. Preferred samples are body fluid samples.

The term “body fluid sample” as used herein refers to a sample of bodily fluid obtained for the purpose of diagnosis, prognosis, classification or evaluation of a subject of interest, such as a patient or transplant donor. In certain embodiments, such a sample may be obtained for the purpose of determining the outcome of an ongoing condition or the effect of a treatment regimen on a condition. Preferred body fluid samples include blood, serum, plasma, cerebrospinal fluid, urine, saliva, sputum, and pleural effusions. In addition, one of skill in the art would realize that certain body fluid samples would be more readily analyzed following a fractionation or purification procedure, for example, separation of whole blood into serum or plasma components. A body fluid sample is obtained “immediately prior to” a procedure if it is obtained within 72 hours of initiating the procedure, and preferably within 48 hours, 24 hours, 18 hours, 12 hours, or 6 hours thereof.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of an assay, most preferably an immunoassay, for a kidney injury marker of the present invention, optionally together with other clinical characteristics, to arrive at a diagnosis (that is, the occurrence or nonoccurrence) of an acute renal injury or ARF for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

Similarly, a prognostic risk signals a probability (“a likelihood”) that a given course or outcome will occur. A level or a change in level of a prognostic indicator, which in turn is associated with an increased probability of morbidity (e.g., worsening renal function, future ARF, or death) is referred to as being “indicative of an increased likelihood” of an adverse outcome in a patient.

IGFBP7 and TIMP-2 Assays

In general, immunoassays are specific binding assay that involve contacting a sample containing or suspected of containing a biomarker of interest with at least one antibody that specifically binds to the biomarker. A signal is then generated indicative of the presence or amount of complexes formed by the binding of polypeptides in the sample to the antibody. The signal is then related to the presence or amount of the biomarker in the sample. Numerous methods and devices are well known to the skilled artisan for the detection and analysis of biomarkers. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, and The Immunoassay Handbook, David Wild, ed. Stockton Press, New York, 1994, each of which is hereby incorporated by reference in its entirety, including all tables, figures and claims.

The assay devices and methods known in the art can utilize labeled molecules in various sandwich, competitive, or non-competitive assay formats, to generate a signal that is related to the presence or amount of the biomarker of interest. Suitable assay formats also include chromatographic, mass spectrographic, and protein “blotting” methods. Additionally, certain methods and devices, such as biosensors and optical immunoassays, may be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing immunoassays. But any suitable immunoassay may be utilized, for example, enzyme-linked immunoassays (ELISA), radioimmunoassays (RIAs), lateral flow assays, competitive binding assays, and the like.

Antibodies or other polypeptides may be immobilized onto a variety of solid supports for use in assays. Solid phases that may be used to immobilize specific binding members include include those developed and/or used as solid phases in solid phase binding assays. Examples of suitable solid phases include membrane filters, cellulose-based papers, beads (including polymeric, latex and paramagnetic particles), glass, silicon wafers, microparticles, nanoparticles, TentaGels, AgroGels, PEGA gels, SPOCC gels, and multiple-well plates. An assay strip could be prepared by coating the antibody or a plurality of antibodies in an array on solid support. This strip could then be dipped into the test sample and then processed quickly through washes and detection steps to generate a measurable signal, such as a colored spot. Antibodies or other polypeptides may be bound to specific zones of assay devices either by conjugating directly to an assay device surface, or by indirect binding. In an example of the later case, antibodies or other polypeptides may be immobilized on particles or other solid supports, and that solid support immobilized to the device surface.

Such assays require methods for detection, and one of the most common methods for quantitation of results is to conjugate a detectable label to a protein or nucleic acid that has affinity for one of the components in the biological system being studied. Detectable labels may include molecules that are themselves detectable (e.g., fluorescent moieties, electrochemical labels, metal chelates, etc.) as well as molecules that may be indirectly detected by production of a detectable reaction product (e.g., enzymes such as horseradish peroxidase, alkaline phosphatase, etc.) or by a specific binding molecule which itself may be detectable (e.g., biotin, digoxigenin, maltose, oligohistidine, 2,4-dintrobenzene, phenylarsenate, ssDNA, dsDNA, etc.).

Preparation of solid phases and detectable label conjugates often comprise the use of chemical cross-linkers. Cross-linking reagents contain at least two reactive groups, and are divided generally into homofunctional cross-linkers (containing identical reactive groups) and heterofunctional cross-linkers (containing non-identical reactive groups). Homobifunctional cross-linkers that couple through amines, sulfhydryls or react non-specifically are available from many commercial sources. Maleimides, alkyl and aryl halides, alpha-haloacyls and pyridyl disulfides are thiol reactive groups. Maleimides, alkyl and aryl halides, and alpha-haloacyls react with sulfhydryls to form thiol ether bonds, while pyridyl disulfides react with sulfhydryls to produce mixed disulfides. The pyridyl disulfide product is cleavable. Imidoesters are also very useful for protein-protein cross-links. A variety of heterobifunctional cross-linkers, each combining different attributes for successful conjugation, are commercially available.

In certain aspects, the present invention provides kits for the analysis of IGFBP7 and/or TIMP-2. The kit comprises reagents for the analysis of at least one test sample which comprise at least one antibody that bind each biomarker being assayed. The kit can also include devices and instructions for performing one or more of the diagnostic and/or prognostic correlations described herein. Preferred kits will comprise an antibody pair for performing a sandwich assay, or a labeled species for performing a competitive assay, for the analyte. Preferably, an antibody pair comprises a first antibody conjugated to a solid phase and a second antibody conjugated to a detectable label, wherein each of the first and second antibodies that bind a kidney injury marker. Most preferably each of the antibodies are monoclonal antibodies. The instructions for use of the kit and performing the correlations can be in the form of labeling, which refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

Antibodies

The term “antibody” as used herein refers to a peptide or polypeptide derived from, modeled after or substantially encoded by an immunoglobulin gene or immunoglobulin genes, or fragments thereof, capable of specifically binding an antigen or epitope. See, e.g. Fundamental Immunology, 3 rd Edition, W. E. Paul, ed., Raven Press, N.Y. (1993); Wilson (1994; J. Immunol. Methods 175:267-273; Yarmush (1992) J. Biochem. Biophys. Methods 25:85-97. The term antibody includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CHl domains; (ii) a F(ab')2 fragment, a bivalent fragment comprising two Fab fragments linked by a disulfide bridge at the hinge region; (iii) a Fd fragment consisting of the VH and CHl domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Antibodies used in the immunoassays described herein preferably specifically bind to a kidney injury marker of the present invention. The term “specifically binds” is not intended to indicate that an antibody binds exclusively to its intended target since, as noted above, an antibody binds to any polypeptide displaying the epitope(s) to which the antibody binds. Rather, an antibody “specifically binds” if its affinity for its intended target is about 5-fold greater when compared to its affinity for a non-target molecule which does not display the appropriate epitope(s). Preferably the affinity of the antibody will be at least about 5 fold, preferably 10 fold, more preferably 25-fold, even more preferably 50-fold, and most preferably 100-fold or more, greater for a target molecule than its affinity for a non-target molecule. In preferred embodiments, Preferred antibodies bind with affinities of at least about 10⁷ M⁻¹, and preferably between about 10⁸ M⁻¹ to about 10⁹ M⁻¹, about 10⁹ M⁻¹ to about 10¹⁰ M⁻¹, or about 10¹⁰ M⁻¹ to about 10¹² M⁻¹ .

Affinity is calculated as K_{d}=k_{off}/kₒₙ (koff is the dissociation rate constant, Kₒₙ is the association rate constant and Kd is the equilibrium constant). Affinity can be determined at equilibrium by measuring the fraction bound (r) of labeled ligand at various concentrations (c). The data are graphed using the Scatchard equation: r/c=K(n−r): where r=moles of bound ligand/mole of receptor at equilibrium; c=free ligand concentration at equilibrium; K =equilibrium association constant; and n=number of ligand binding sites per receptor molecule. By graphical analysis, r/c is plotted on the Y-axis versus r on the X-axis, thus producing a Scatchard plot. Antibody affinity measurement by Scatchard analysis is well known in the art. See, e.g., van Erp et al., J. Immunoassay 12: 425-43, 1991; Nelson and Griswold, Comput. Methods Programs Biomed. 27: 65-8, 1988.

The term “epitope” refers to an antigenic determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Numerous publications discuss the use of phage display technology to produce and screen libraries of polypeptides for binding to a selected analyte. See, e.g, Cwirla et al., Proc. Natl. Acad. Sci. USA 87, 6378-82, 1990; Devlin et al., Science 249, 404-6, 1990, Scott and Smith, Science 249, 386-88, 1990; and Ladner et al., U.S. Pat. No. 5,571,698. A basic concept of phage display methods is the establishment of a physical association between DNA encoding a polypeptide to be screened and the polypeptide. This physical association is provided by the phage particle, which displays a polypeptide as part of a capsid enclosing the phage genome which encodes the polypeptide. The establishment of a physical association between polypeptides and their genetic material allows simultaneous mass screening of very large numbers of phage bearing different polypeptides. Phage displaying a polypeptide with affinity to a target bind to the target and these phage are enriched by affinity screening to the target. The identity of polypeptides displayed from these phage can be determined from their respective genomes. Using these methods a polypeptide identified as having a binding affinity for a desired target can then be synthesized in bulk by conventional means. See, e.g., U.S. Pat. No. 6,057,098, which is hereby incorporated in its entirety, including all tables, figures, and claims.

The antibodies that are generated by these methods may then be selected by first screening for affinity and specificity with the purified polypeptide of interest and, if required, comparing the results to the affinity and specificity of the antibodies with polypeptides that are desired to be excluded from binding. The screening procedure can involve immobilization of the purified polypeptides in separate wells of microtiter plates. The solution containing a potential antibody or groups of antibodies is then placed into the respective microtiter wells and incubated for about 30 min to 2 h. The microtiter wells are then washed and a labeled secondary antibody (for example, an anti-mouse antibody conjugated to alkaline phosphatase if the raised antibodies are mouse antibodies) is added to the wells and incubated for about 30 min and then washed. Substrate is added to the wells and a color reaction will appear where antibody to the immobilized polypeptide(s) are present.

The antibodies so identified may then be further analyzed for affinity and specificity in the assay design selected. In the development of immunoassays for a target protein, the purified target protein acts as a standard with which to judge the sensitivity and specificity of the immunoassay using the antibodies that have been selected. Because the binding affinity of various antibodies may differ; certain antibody pairs (e.g., in sandwich assays) may interfere with one another sterically, etc., assay performance of an antibody may be a more important measure than absolute affinity and specificity of an antibody.

While the present application describes antibody-based binding assays in detail, alternatives to antibodies as binding species in assays are well known in the art. These include receptors for a particular target, aptamers, etc. Aptamers are oligonucleic acid or peptide molecules that bind to a specific target molecule. Aptamers are usually created by selecting them from a large random sequence pool, but natural aptamers also exist. High-affinity aptamers containing modified nucleotides conferring improved characteristics on the ligand, such as improved in vivo stability or improved delivery characteristics. Examples of such modifications include chemical substitutions at the ribose and/or phosphate and/or base positions, and may include amino acid side chain functionalities.

Assay Correlations

The term “correlating” as used herein in reference to the use of biomarkers refers to comparing the presence or amount of the biomarker(s) in a patient to its presence or amount in persons known to suffer from, or known to be at risk of, a given condition; or in persons known to be free of a given condition. Often, this takes the form of comparing an assay result in the form of a biomarker concentration to a predetermined threshold selected to be indicative of the occurrence or nonoccurrence of a disease or the likelihood of some future outcome.

Selecting a diagnostic threshold involves, among other things, consideration of the probability of disease, distribution of true and false diagnoses at different test thresholds, and estimates of the consequences of treatment (or a failure to treat) based on the diagnosis. For example, when considering administering a specific therapy which is highly efficacious and has a low level of risk, few tests are needed because clinicians can accept substantial diagnostic uncertainty. On the other hand, in situations where treatment options are less effective and more risky, clinicians often need a higher degree of diagnostic certainty. Thus, cost/benefit analysis is involved in selecting a diagnostic threshold.

Suitable thresholds may be determined in a variety of ways. For example, one recommended diagnostic threshold for the diagnosis of acute myocardial infarction using cardiac troponin is the 97.5th percentile of the concentration seen in a normal population. Another method may be to look at serial samples from the same patient, where a prior “baseline” result is used to monitor for temporal changes in a biomarker level.

Population studies may also be used to select a decision threshold. Reciever Operating Characteristic (“ROC”) arose from the field of signal dectection therory developed during World War II for the analysis of radar images, and ROC analysis is often used to select a threshold able to best distinguish a “diseased” subpopulation from a “nondiseased” subpopulation. A false positive in this case occurs when the person tests positive, but actually does not have the disease. A false negative, on the other hand, occurs when the person tests negative, suggesting they are healthy, when they actually do have the disease. To draw a ROC curve, the true positive rate (TPR) and false positive rate (FPR) are determined as the decision threshold is varied continuously. Since TPR is equivalent with sensitivity and FPR is equal to 1—specificity, the ROC graph is sometimes called the sensitivity vs (1—specificity) plot. A perfect test will have an area under the ROC curve of 1.0; a random test will have an area of 0.5. A threshold is selected to provide an acceptable level of specificity and sensitivity.

In this context, “diseased” is meant to refer to a population having one characteristic (the presence of a disease or condition or the occurrence of some outcome) and “nondiseased” is meant to refer to a population lacking the characteristic. While a single decision threshold is the simplest application of such a method, multiple decision thresholds may be used. For example, below a first threshold, the absence of disease may be assigned with relatively high confidence, and above a second threshold the presence of disease may also be assigned with relatively high confidence. Between the two thresholds may be considered indeterminate. This is meant to be exemplary in nature only.

In addition to threshold comparisons, other methods for correlating assay results to a patient classification (occurrence or nonoccurrence of disease, likelihood of an outcome, etc.) include decision trees, rule sets, Bayesian methods, and neural network methods. These methods can produce probability values representing the degree to which a subject belongs to one classification out of a plurality of classifications.

Measures of test accuracy may be obtained as described in Fischer et al., Intensive Care Med. 29: 1043-51, 2003, and used to determine the effectiveness of a given biomarker. These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

As discussed above, suitable tests may exhibit one or more of the following results on these various measures: a specificity of greater than 0.5, preferably at least 0.6, more preferably at least 0.7, still more preferably at least 0.8, even more preferably at least 0.9 and most preferably at least 0.95, with a corresponding sensitivity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; a sensitivity of greater than 0.5, preferably at least 0.6, more preferably at least 0.7, still more preferably at least 0.8, even more preferably at least 0.9 and most preferably at least 0.95, with a corresponding specificity greater than 0.2, preferably greater than 0.3, more preferably greater than 0.4, still more preferably at least 0.5, even more preferably 0.6, yet more preferably greater than 0.7, still more preferably greater than 0.8, more preferably greater than 0.9, and most preferably greater than 0.95; at least 75% sensitivity, combined with at least 75% specificity; a ROC curve area of greater than 0.5, preferably at least 0.6, more preferably 0.7, still more preferably at least 0.8, even more preferably at least 0.9, and most preferably at least 0.95; an odds ratio different from 1, preferably at least about 2 or more or about 0.5 or less, more preferably at least about 3 or more or about 0.33 or less, still more preferably at least about 4 or more or about 0.25 or less, even more preferably at least about 5 or more or about 0.2 or less, and most preferably at least about 10 or more or about 0.1 or less; a positive likelihood ratio (calculated as sensitivity/(1-specificity)) of greater than 1, at least 2, more preferably at least 3, still more preferably at least 5, and most preferably at least 10; and or a negative likelihood ratio (calculated as (1-sensitivity)/specificity) of less than 1, less than or equal to 0.5, more preferably less than or equal to 0.3, and most preferably less than or equal to 0.1

Clinical indicia which may be combined with the kidney injury marker assay result(s) of the present invention includes demographic information (e.g., weight, sex, age, race), medical history (e.g., family history, type of surgery, pre-existing disease such as aneurism, congestive heart failure, preeclampsia, eclampsia, diabetes mellitus, hypertension, coronary artery disease, proteinuria, renal insufficiency, or sepsis, type of toxin exposure such as NSAIDs, cyclosporines, tacrolimus, aminoglycosides, foscarnet, ethylene glycol, hemoglobin, myoglobin, ifosfamide, heavy metals, methotrexate, radiopaque contrast agents, or streptozotocin), clinical variables (e.g., blood pressure, temperature, respiration rate), risk scores (APACHE score, PREDICT score, TIMI Risk Score for UA/NSTEMI, Framingham Risk Score), a urine total protein measurement, a glomerular filtration rate, an estimated glomerular filtration rate, a urine production rate, a serum or plasma creatinine concentration, a renal papillary antigen 1 (RPA1) measurement; a renal papillary antigen 2 (RPA2) measurement; a urine creatinine concentration, a fractional excretion of sodium, a urine sodium concentration, a urine creatinine to serum or plasma creatinine ratio, a urine specific gravity, a urine osmolality, a urine urea nitrogen to plasma urea nitrogen ratio, a plasma BUN to creatnine ratio, and/or a renal failure index calculated as urine sodium/(urine creatinine/plasma creatinine). Other measures of renal function which may be combined in the methods of the present invention are described hereinafter and in Harrison's Principles of Internal Medicine, 17^{th} Ed., McGraw Hill, New York, pages 1741-1830, and Current Medical Diagnosis & Treatment 2008, 47^{th} Ed, McGraw Hill, New York, pages 785-815, each of which are hereby incorporated by reference in their entirety.

Combining assay results/clinical indicia in this manner can comprise the use of multivariate logistical regression, loglinear modeling, neural network analysis, n-of-m analysis, decision tree analysis, etc. This list is not meant to be limiting.

Diagnosis of Acute Renal Failure

As noted above, the terms “acute renal (or kidney) injury” and “acute renal (or kidney) failure” as used herein are defined in part in terms of changes in serum creatinine from a baseline value. Most definitions of ARF have common elements, including the use of serum creatinine and, often, urine output. Patients may present with renal dysfunction without an available baseline measure of renal function for use in this comparison. In such an event, one may estimate a baseline serum creatinine value by assuming the patient initially had a normal GFR. Glomerular filtration rate (GFR) is the volume of fluid filtered from the renal (kidney) glomerular capillaries into the Bowman's capsule per unit time. Glomerular filtration rate (GFR) can be calculated by measuring any chemical that has a steady level in the blood, and is freely filtered but neither reabsorbed nor secreted by the kidneys. GFR is typically expressed in units of ml/min:

$$GFR = \frac{Urine Concentration \times Urine Flow}{Plasma Concentration}$$

By normalizing the GFR to the body surface area, a GFR of approximately 75-100 ml/min per 1.73 m² can be assumed. The rate therefore measured is the quantity of the substance in the urine that originated from a calculable volume of blood.

There are several different techniques used to calculate or estimate the glomerular filtration rate (GFR or eGFR). In clinical practice, however, creatinine clearance is used to measure GFR. Creatinine is produced naturally by the body (creatinine is a metabolite of creatine, which is found in muscle). It is freely filtered by the glomerulus, but also actively secreted by the renal tubules in very small amounts such that creatinine clearance overestimates actual GFR by 10-20%. This margin of error is acceptable considering the ease with which creatinine clearance is measured.

Creatinine clearance (CCr) can be calculated if values for creatinine's urine concentration (U_{Cr}), urine flow rate (V), and creatinine's plasma concentration (P_{Cr}) are known. Since the product of urine concentration and urine flow rate yields creatinine's excretion rate, creatinine clearance is also said to be its excretion rate (U_{Cr}×V) divided by its plasma concentration. This is commonly represented mathematically as:

$${C}_{Cr} = \frac{{U}_{Cr} \times V}{{P}_{Cr}}$$

Commonly a 24 hour urine collection is undertaken, from empty-bladder one morning to the contents of the bladder the following morning, with a comparative blood test then taken:

$${C}_{Cr} = \frac{{U}_{Cr} \times 24 \text{-} hour volume}{{P}_{Cr} \times 24 \times 60 mins}$$

To allow comparison of results between people of different sizes, the CCr is often corrected for the body surface area (BSA) and expressed compared to the average sized man as ml/min/1.73 m2. While most adults have a BSA that approaches 1.7 (1.6-1.9), extremely obese or slim patients should have their CCr corrected for their actual BSA:

$${C}_{Cr - corrected} = \frac{{C}_{Cr} \times 1.73}{BSA}$$

The accuracy of a creatinine clearance measurement (even when collection is complete) is limited because as glomerular filtration rate (GFR) falls creatinine secretion is increased, and thus the rise in serum creatinine is less. Thus, creatinine excretion is much greater than the filtered load, resulting in a potentially large overestimation of the GFR (as much as a twofold difference). However, for clinical purposes it is important to determine whether renal function is stable or getting worse or better. This is often determined by monitoring serum creatinine alone. Like creatinine clearance, the serum creatinine will not be an accurate reflection of GFR in the non-steady-state condition of ARF. Nonetheless, the degree to which serum creatinine changes from baseline will reflect the change in GFR. Serum creatinine is readily and easily measured and it is specific for renal function.

For purposes of determining urine output on a Urine output on a mL/kg/hr basis, hourly urine collection and measurement is adequate. In the case where, for example, only a cumulative 24-h output was available and no patient weights are provided, minor modifications of the RIFLE urine output criteria have been described. For example, Bagshaw et al., Nephrol. Dial. Transplant. 23: 1203-1210, 2008, assumes an average patient weight of 70 kg, and patients are assigned a RIFLE classification based on the following: <35 mL/h (Risk), <21 mL/h (Injury) or <4 mL/h (Failure).

Selecting a Treatment Regimen

Once a diagnosis is obtained, the clinician can readily select a treatment regimen that is compatible with the diagnosis, such as initiating renal replacement therapy, withdrawing delivery of compounds that are known to be damaging to the kidney, kidney transplantation, delaying or avoiding procedures that are known to be damaging to the kidney, modifying diuretic administration, initiating goal directed therapy, etc. The skilled artisan is aware of appropriate treatments for numerous diseases discussed in relation to the methods of diagnosis described herein. See, e.g., Merck Manual of Diagnosis and Therapy, 17th Ed. Merck Research Laboratories, Whitehouse Station, N.J., 1999. In addition, since the methods and compositions described herein provide prognostic information, the markers of the present invention may be used to monitor a course of treatment. For example, improved or worsened prognostic state may indicate that a particular treatment is or is not efficacious.

The distinction between prerenal AKI and instrinsic AKI is an important clinical assessment that directs the therapeutic intervention(s). Patients who are prerenal need therapies directed at hemodynamics to improve renal blood flow. These therapies are often involve inotropes, intravenous fluids and/or vasopressors. Each of these interventions have potential side effects (e.g. arrhythmias, volume overload, vasoconstriction) and would not be advisable to implement these therapies if they are not destined to improve renal function. Thus, the distinction between prerenal AKI and intrinsic AKI helps determine the therapy which should be prescribed. If prerenal AKI is not present, therapy is directed at mitigating AKI and providing supportive care.

Prerenal acute renal failure occurs when a sudden reduction in blood flow to the kidney camera (renal hypoperfusion) causes a loss of kidney function. Causes can include low blood volume, low blood pressure, shunting of blood from the kidney, heart failure, and local changes to the blood vessels supplying the kidney. In prerenal acute renal failure, there is nothing wrong with the kidney itself. Treatment focuses on correcting the cause of the prerenal acute renal failure.

In prerenal AKI without fluid overload, administration of intravenous fluids is typically the first step to improve renal function. This is particularly used in patients in whom prerenal AKI develops as the result of intravascular volume depletion in order to restore normal circulating blood volume. Volume status may be monitored to avoid over- or under-replacement of fluid as described herein. Fluids with colloidal particles such as albumin may be preferred over simple saline infusion. In a prerenal condition wherein the forward flow is compromised, drugs directed at augmenting cardiac output are typically employed.

In patients with congestive heart failure in whom AKI has developed as a result of excessive diuresis, withholding of diuretics and cautious volume replacement may be sufficient to restore kidney function. Inotropes such as norepinephrine and dobutamine may be given to improve cardiac output and hence renal perfusion.

Hospitalized fluid overload patients are typically treated with fluid restriction, IV diuretics, inotropes (e.g., milrinone or dobutamine) and combination therapies. The loop diuretic furosemide is the most frequently prescribed diuretic for treatment of volume overload in HF. Initial oral doses of 20 to 40 mg once a day should be administered to patients with dyspnea on exertion and signs of volume overload who do not have indications for acute hospitalization. Severe overload and pulmonary edema are indications for hospitalization and intravenous furosemide. Some patients with mild HF can be treated effectively with thiazide diuretics. Those who have persistent volume overload on a thiazide diuretic should be switched to an oral loop diuretic. In patients with severe kidney injury, diuretics may not result in significant diuresis. Ultrafiltration, also called aquapheresis, may be used to treat fluid overload in such cases.

In contrast to prerenal AKI, the main goal of treatment of acute tubular necrosis (ATN) is to prevent further injury to the kidney. Ischemic ATN can be caused when the kidneys are not sufficiently perfused for a long period of time (e.g. due to renal artery stenosis) or by shock. Sepsis causes 30% to 70% of deaths in patients with ATN; therefore, avoidance of intravenous lines, bladder catheters, and respirators is recommended. Because septic patients are vasodilated, large volumes of administered fluid accumulate in the lung interstitium of these patients. Extracellular fluid volume should be assessed promptly, and repletion of any deficit should be initiated promptly. Hemodynamic status should be modified by appropriate fluid therapy, giving vasopressors and/or inotropes and treating any underlying sepsis. All possible nephrotoxic drugs should be stopped. In addition, doses of all medications that are eliminated by the kidney should be adjusted.

Renal replacement therapy refers to therapy that replaces the normal blood-filtering function of the kidneys. Various types of RRT are used by clinicians, including the following:
continuous renal replacement therapy (CRRT)
   continuous hemodialysis (CHD)
      continuous arteriovenous hemodialysis (CAVHD)
      continuous venovenous hemodialysis (CVVHD)
   continuous hemofiltration (CHF)
      continuous arteriovenous hemofiltration (CAVH or CAVHF)
      continuous venovenous hemofiltration (CVVH or CVVHF)
   continuous hemodiafiltration (CHDF)
      continuous arteriovenous hemodiafiltration (CAVHDF)
      continuous venovenous hemodiafiltration (CVVHDF)
intermittent renal replacement therapy (IRRT)
   intermittent hemodialysis (IHD)
      intermittent venovenous hemodialysis (IVVHD)
   intermittent hemofiltration (IHF)
      intermittent venovenous hemofiltration (IVVH or IVVHF)
   intermittent hemodiafiltration (IHDF)

Acute dialysis-dependent renal failure is a common problem in the intensive care unit (ICU) and, despite significant improvements in the care of critically ill patients, the mortality from this complication remains over 50%. The development of renal failure is an independent predictor of mortality in this patient population.

The precise timing of RRT initiation is usually a matter of clinical judgment. The classic indications for dialysis include:

diuretic resistant pulmonary edema

hyperkalemia (refractory to medical therapy)

metabolic acidosis (refractory to medical therapy)

uremic complications (pericarditis, encephalopathy, bleeding)

dialyzable intoxications (eg, lithium, toxic alcohols, and salicylates).

While many of these indications are typically used in the setting of chronic renal failure, the consequences of these complications are likely to be more severe in critically ill patients; therefore, there has been a growing trend to start dialysis prior to the development of these indications. Delays in the initiation of treatment have often been based on a concern that dialysis itself may delay recovery of renal function.

IGFBP7 and TIMP-2 have been described for risk assessment of AKI. Kellum and Chawla, Neohrol. Dial.. Transplant 31(1):16-22, 2016. The present invention demonstrates that these biomarkers can also be used for assessing whether kidney function is “under stress” for purposes of managing the administration of renal replacement therapy to minimize further stress that will lead to additional renal damage.

CRRT is any renal replacement therapy that is intended to be applied for 24 h per day in an ICU. The term CRRT describes a variety of blood purification techniques, which may differ significantly according to the mechanism of solute transport, the type of membrane, the presence or absence of dialysate solution, and the type of vascular access. CRRT provides slower solute clearance per unit time as compared with intermittent therapies but over 24 h may even exceed clearances with IHD. The choice of CRRT is thought to provide better hemodynamic tolerability, more efficient solute clearance, better control of intravascular volume, and better clearance of middle and large molecular weight substances relative to intermittent dialysis. Pannu and Gibney, Ther. Clin. Risk. Manag. 1: 141-50, 2005, which is hereby incorporated by reference in its entirety.

Hypotension is one of the most common complications associated with intermittent hemodialysis, occurring in approximately 20%-30% of all treatments. Some of the causes are dialysis specific, such as excessive or rapid volume removal, changes in plasma osmolality, and autonomic dysfunction. In critically ill patients who may be hemodynamically unstable, it would be desirable to minimize this complication, as it may lead to further organ ischemia and injury. The risk scores of the present invention may be used to determine if a shift is necessary between, for example, intermittent hemodialysis, and a method of renal replacement therapy that produces less renal stress. In this regard, when the risk score is elevated above the applicable threshold, one may reduce the rate or amount of fluid being removed. Additionally, the clearance rate of small solutes (e.g., urea) is slower per unit time with CRRT (17 mL/min vs more than 160 mL/min with intermittent hemodialysis).

One skilled in the art readily appreciates that the present invention is well adapted to carry out the objects and obtain the ends and advantages mentioned, as well as those inherent therein. The examples provided herein are representative of preferred embodiments, are exemplary, and are not intended as limitations on the scope of the invention.

### EXAMPLE 1

### Longer Duration of RRT for Patients with Elevated Biomarkers

ICU patients with acute kidney injury (AKI) and receiving renal replacement therapy (RRT) were included in the analysis. A urine sample was collected from each patient during RRT and within 48 hours after initiation of RRT. TIMP2, IGFBP7, and TIMP2×IGBFP7 (multiplication of the concentrations of the two biomarkers) were measured in the urine samples by immunoassay with the NephroCheck® Test kit on the Astute140® Meter. Patients were divided into two groups by their biomarker concentrations, being either less than or equal to or greater than the specified threshold. The range and median of the number of days on RRT, length of hospital stay, and length of ICU stay were determined for each patient group. Patients with biomarker concentrations greater than the threshold received RRT for more days and had a longer length of stay in the hospital and in the ICU than patients with biomarker concentrations less than or equal to the threshold.

| | [TIMP2] × | Endpoint | | | |
|---|---|---|---|---|---|
| | [IGFBP7], | | | | Kruskal_Wallis |
| Endpoint | (ng/mL)²/1000 | n | Range | Median(IQR) | P-value |
| Days on RRT | ≤2 | 19 | 1.00 to 9.00 | 3.00(2.00-4.00) | 0.007 |
| | >2 | 24 | 1.00 to 9.00 | 5.00(4.00-6.00) | |
| Hospital Length | ≤2 | 19 | 3.00 to 95.00 | 21.00(9.00-47.00) | 0.561 |
| of Stay | >2 | 23 | 2.00 to 69.00 | 32.00(17.00-45.00) | |
| ICU Length | ≤2 | 18 | 3.00 to 95.00 | 9.50(6.00-19.00) | 0.185 |
| of Stay | >2 | 21 | 3.00 to 50.00 | 19.00(7.00-26.00) | |

| | | Endpoint | | | |
|---|---|---|---|---|---|
| | [IGFBP7], | | | | Kruskal_Wallis |
| Endpoint | ng/mL | n | Range | Median(IQR) | P-value |
| Days on RRT | ≤150 | 18 | 1.00 to 9.00 | 3.50(2.00-5.00) | 0.121 |
| | >150 | 25 | 1.00 to 9.00 | 5.00(3.00-6.00) | |
| Hospital Length | ≤150 | 18 | 3.00 to 95.00 | 20.00(9.00-45.00) | 0.438 |
| of Stay | >150 | 24 | 2.00 to 69.00 | 31.50(18.00-49.50) | |
| ICU Length | ≤150 | 18 | 3.00 to 95.00 | 9.50(6.00-19.00) | 0.171 |
| of Stay | >150 | 21 | 3.00 to 50.00 | 20.00(7.00-26.00) | |

| | | Endpoint | | | |
|---|---|---|---|---|---|
| | [TIMP2], | | | | Kruskal_Wallis |
| Endpoint | ng/mL | n | Range | Median(IQR) | P-value |
| Days on RRT | ≤12 | 17 | 1.00 to 9.00 | 3.00(2.00-4.00) | 0.002 |
| | >12 | 26 | 1.00 to 9.00 | 5.00(4.00-6.00) | |
| Hospital Length | ≤12 | 17 | 3.00 to 95.00 | 23.00(14.00-47.00) | 0.980 |
| of Stay | >12 | 25 | 2.00 to 69.00 | 31.00(14.00-45.00) | |
| ICU Length | ≤12 | 16 | 3.00 to 95.00 | 11.50(5.50-23.00) | 0.501 |
| of Stay | >12 | 23 | 3.00 to 50.00 | 18.00(6.00-26.00) | |

### EXAMPLE 2

### Use of Biomarkers for Choosing RRT Modality

A 65 year-old male is admitted to the intensive care unit (ICU) after presenting to the emergency department with a diagnosis of a severe, community acquired pneumonia. Due to worsening respiratory insufficiency and an inability to maintain adequate oxygenation, he is intubated and placed on mechanical ventilation. He also is noted to have a low blood pressure and received several liters of intravenous (IV) crystalloid intravenous fluid for volume resuscitation. He does not respond and as a result, vasopressor therapy is started to maintain systemic blood pressure. He is also pancultured and placed on broad-spectrum antimicrobial therapy.

His urine output remains persistently below than 0.3 mL/kg/hr since his admission despite the aggressive volume resuscitation that he receives and his serum creatinine rises from an admission level of 1.3 mg/dL to 5.1 mg/dL, suggesting AKI stage III. He requires significant positive pressure ventilatory support, including elevated FiO2 and PEEP. Of note, his pulmonary compliance is decreased, his central venous pressure is persistently elevated, and he is becoming increasingly edematous, all of which suggest significant total body fluid overload. He is evaluated with a transthoracic echo (TTE) to assess his cardiac function and performance and also, shortly after admission to the ICU, he has a central venous line (CVL) placed for intravenous assess and for assessment of central venous pressure (CVP), which remains consistently elevated.

Based on his clinical status, the patient is a candidate for RRT. A urine sample is collected for measurement of [TIMP2]×[IGFBP7]. The [TIMP2]×[IGFBP7] is >2.0, indicating high levels of kidney stress. The elevated [TIMP2]×[IGFBP7] level (high kidney stress) indicates the patient's kidneys have a low tolerance for the hemodynamic instability and/or other systemic physiological derangements associated with the patient's condition. In addition, the elevated [TIMP2]×[IGFBP7] level indicates risk of a prolonged course of RRT. Therefore, the clinical team selects continuous renal replacement therapy (rather than intermittent renal replacement therapy), which is recommended in situations in which shifts in fluid balance and metabolic fluctuations are poorly tolerated.

While the invention has been described and exemplified in sufficient detail for those skilled in this art to make and use it, various alternatives, modifications, and improvements should be apparent without departing from the spirit and scope of the invention. The examples provided herein are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication is specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

Other embodiments are set forth within the following claims.

We claim:

1. A method for treating renal stress in a subject in need of renal replacement therapy, comprising:
calculating a risk score which is (i) a urinary concentration of IGFBP7 (insulin-like growth factor-binding protein 7), (ii) a urinary concentration of TIMP-2 (tissue inhibitor of metalloproteinase 2), or (iii) a composite of a urinary concentration of IGFBP7 and a urinary concentration of TIMP-2, by measuring an IGFBP7 concentration and/or a TIMP-2 concentration in a urine sample obtained from the subject to provide the risk score;
comparing the risk score to a risk score threshold value, wherein when the risk score is above the risk score threshold value the subject is determined to be in renal stress; and
if the comparing step indicates that the subject is in renal stress, treating the subject with a method of renal replacement therapy that produces less renal stress relative to treatment with intermittent hemodialysis.

2. A method according to claim 1, wherein the method of renal replacement therapy that produces less renal stress relative to treatment with intermittent hemodialysis is continuous renal replacement therapy or prolonged intermittent renal replacement therapy (PIRRT).

3. A method according to claim 1 or 2, wherein the risk score is calculated by multiplication of the concentrations of IGFBP7 and TIMP-2.

4. The method according to claim 3, wherein the risk score is [TIMP-2]×[IGFBP7]/1000, where the concentrations of IGFBP7 and TIMP-2 are each measured in ng/mL.

5. The method according to claim 4, wherein the threshold is about 2.0.

6. A method according to one of claims 1-5, wherein the urinary concentration of IGFBP7 and/or the urinary concentration of TIMP-2 are measured by introducing the urine sample obtained from the subject into an immunoassay instrument; wherein the immunoassay instrument comprises a solid phase, and one or both of an IGFBP7 antibody immobilized at a first location on the solid phase and a TIMP-2 antibody immobilized at a second location on the solid phase; wherein the instrument causes the urine sample to contact one or both of the first location and the second location;
wherein the instrument measures the amount of IGFBP7 which binds to the IGFBP7 antibody immobilized at the first location and determines therefrom the concentration of IGFBP7 in the urine sample; and/or wherein the instrument measures the amount of TIMP-2 which binds to the TIMP-2 antibody immobilized at the second location and determines therefrom the concentration of TIMP-2 in the urine sample;
wherein the instrument optionally mathematically combines the concentration of IGFBP7 and the concentration of TIMP-2 in the urine sample into the risk score; and
wherein the instrument reports the risk score in a human readable form.

7. A method according to claim 6, wherein the urine sample obtained from the subject is further contacted with a second IGFBP7 antibody conjugated to detectable label and a second TIMP-2 antibody conjugated to detectable label; wherein first sandwich complexes are formed between the IGFBP7 antibody, IGFBP7 present in the urine sample, and the second IGFBP7 antibody; wherein second sandwich complexes are formed between the TIMP-2 antibody, TIMP-2 present in the urine sample, and the second TIMP-2 antibody; wherein the amount of IGFBP7 which binds to the IGFBP7 antibody is determined by the instrument detecting the detectable label bound at the first location; and wherein the amount of TIMP-2 which binds to the TIMP-2 antibody is determined by the instrument detecting the detectable label bound at the second location.

8. A method according to one of claims 1-7, wherein the subject is an intensive care unit patient.

9. A method according to one of claims 1-8, wherein the patient is in acute renal failure.

10. A method according to one of claims 1-9, wherein the subject has sepsis.

11. A method according to one of claims 1-9, wherein the subject is recovering from surgery.

12. A method according to one of claims 1-11, wherein the subject is undergoing renal replacement therapy at the time the urine sample is obtained from the subject to provide the risk score.

13. A method according to claim 12, wherein the risk score is used to monitor ongoing renal replacement therapy, wherein if the risk score is above the threshold, the rate or amount of fluid volume being removed from the subject by the ongoing renal replacement therapy is reduced, and/or the clearance rate of solutes by the ongoing renal replacement therapy is reduced.

14. A method according to claim 12, wherein the risk score is used to monitor ongoing renal replacement therapy, wherein if the risk score is above the threshold, the ongoing renal replacement therapy protocol is adjusted to reduce hypotensive effects associated with the ongoing renal replacement therapy or dose.
